(19) United States
(12) Patent Application Publication
(10) Pub. No.: US 2020/0095528 A1
(43) Pub. Date: Mar. 26, 2020

(54) ECOSYSTEM FOR METHANE CONVERSION AND EMISSIONS REDUCTION

(71) Applicants: Albert James Lacaze, Potomac, MD (US); Jack Treston Goehrung, Rockville, MD (US); Sarkis Haig Pakchanian, Gaithersburg, MD (US); Brendan Patrick O'Boyle, Rockville, MD (US); Gabriel Luis Salazar Moron, Derwood, MD (US); Holden Ryan Leighton, Rockville, MD (US); Jackson Kendricks, Montgomery Village, MD (US)

(72) Inventors: Albert James Lacaze, Potomac, MD (US); Jack Treston Goehrung, Rockville, MD (US); Sarkis Haig Pakchanian, Gaithersburg, MD (US); Brendan Patrick O'Boyle, Rockville, MD (US); Gabriel Luis Salazar Moron, Derwood, MD (US); Holden Ryan Leighton, Rockville, MD (US); Jackson Kendricks, Montgomery Village, MD (US)

(21) Appl. No.: 16/140,840

(22) Filed: Sep. 25, 2018

(51) Int. Cl. C12M 1/00; C01B 32/50; A01G 33/00; A01K 1/00

(52) U.S. Cl. CPC C12M 23/18; A01K 1/0052; A01G 33/00; C01B 32/50

(57) ABSTRACT

The ecosystem for methane conversion and emissions reduction disclosed herein presents a low-cost solution to the global methane accumulation problem. Ruminants generate gases as part of their digestive process, including methane, and the negative effects of methane in our atmosphere are well known. Many attempts are underway to use diet modifiers to lessen the amount of methane created and therefore minimize the effect. The presented invention takes a different approach. A methane converter is used in conjunction with a closed system to first transform the methane into the less damaging CO₂ and then to use the CO₂ for feeding algae growth. Methane at concentration levels in the enclosed habitat are maintained low to minimize the possibility of explosion, and the conversion from methane to CO₂ is triggered by heating the low concentration methane with a heated surface or small flame.

### CROSS REFERENCE TO RELATED PATENT APPLICATIONS

The present application makes no reference to any other related filed patent applications.

### STATEMENT REGARDING FEDERAL SPONSORSHIP

No part of this invention was a result of any federally sponsored research.

### TECHNICAL FIELD OF THE INVENTION

The present invention relates in general to emissions reduction, and, more specifically, to an ecosystem for methane conversion and emissions reduction.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Methane, a chemical compound with the chemical formula CH₄, is the main constituent of natural gas and accounts for 20% of the total radiative forcing from all of the globally mixed greenhouse gases. The concentration of methane in Earth's atmosphere has increased about 150% since 1750, and such an increase has demonstrable and known negative effects on global climate. One indication of such effects is the global warming potential (GWP) of methane, which is 86, compared to that of carbon dioxide (CO₂), which is 1, within the first two decades after its generation.

Ruminants, mammals able to acquire nutrients from plant-based food by fermenting it in a specialized stomach prior to digestion, generate methane and other gases as a byproduct of this digestive process. Due to the extensive and increasing use of ruminants in food production, and, thus, their increasing production of methane, research is being conducted into dietary modifications for ruminants that would reduce the amount of methane they discharge. Studies have shown that increasing the level of dietary fat may affect methane production, namely that a diet of crushed oilseeds or dried corn distilled grain reduces the amount of methane released by up to 20%. Increasing the concentration of grain in the feed further reduces the amount of methane produced, but is limited by the dietary needs of the ruminants. Other research has shown that adding seaweed to ruminant diets can reduce the production of methane by more than 70%, or that vaccines may be applied to eliminate the methane-producing microbes in ruminant systems. None of these methods, though, have proven to be commercially viable with current technologies.

Therefore, a need exists in the field for an ecosystem for methane conversion and emissions reduction that can be implemented as a low-cost solution to increasing methane concentrations in the atmosphere.

### BRIEF SUMMARY OF THE INVENTION

To minimize the limitations in the prior art, and to minimize other limitations that will be apparent upon reading and understanding the present specification, the present invention describes an ecosystem for methane conversion and emissions reduction.

It is an objective of the present invention to provide an ecosystem that may comprise a plurality of ruminants.

It is another objective of the present invention to provide an ecosystem that may comprise an enclosed facility.

It is another objective of the present invention to provide an ecosystem that may comprise a methane convertor.

It is another objective of the present invention to provide an ecosystem that may comprise an algae pond.

It is another objective of the present invention to provide an ecosystem that may comprise a closed system.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates an overview of an ecosystem for methane conversion and emissions reduction, as contemplated by the present disclosure.

FIG. 2 illustrates an overview of a methane convertor used in an ecosystem for methane conversion and emissions reduction, as contemplated by the present disclosure.

FIG. 3 illustrates a process by which a methane convertor used in an ecosystem for methane conversion and emissions reduction operates, as contemplated by the present disclosure.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The present invention relates in general to emissions reduction, and, more specifically, to an ecosystem for methane conversion and emissions reduction. As contemplated by the present disclosure, the ecosystem comprises a plurality of ruminants, an enclosed facility, a methane convertor, and an algae pond.

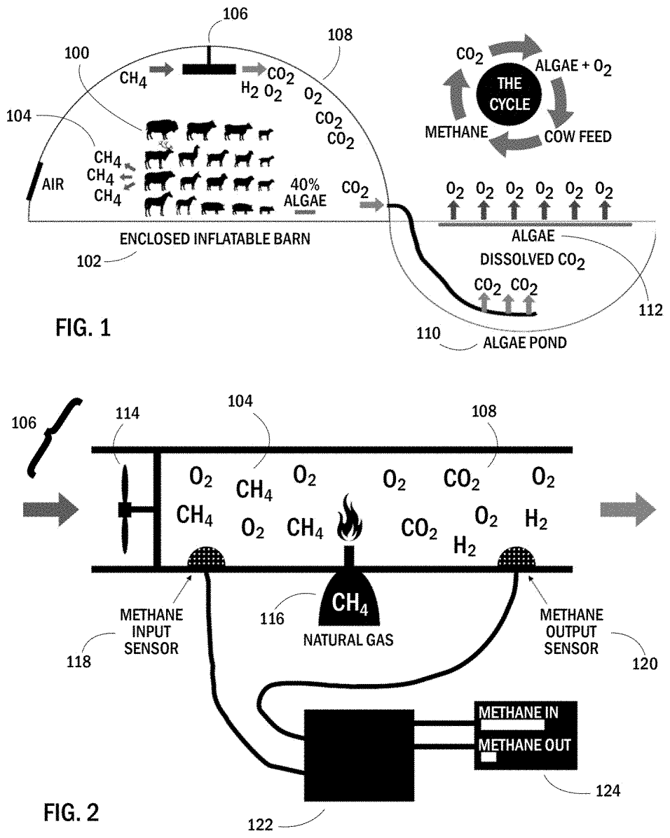

The illustration of FIG. 1 illustrates an overview of an ecosystem for methane conversion and emissions reduction. The plurality of ruminants 100 are housed in an enclosed facility 102 such that the methane produced 104 is retained within a known area. The methane produced 104 is circulated through a methane convertor 106, which converts the methane produced 104 into its products of combustion 108, namely carbon dioxide (CO₂) and water (H₂O). The products of combustion 108 are then transported into an algae pond 110 where they are dissolved into the water of the pond and, thus, fed to the algae 112. The algae 112 is grown by this feeding and, when ready to harvest, is fed to the plurality of ruminants 100 to complete the ecosystem.

Studies have shown that ruminants, specifically cows, fed with algae demonstrate a reduced need for antibiotics and produce higher quality milk and protein rich in vitamins. The beef from these cows may further be considered organic, since it is grown without antibiotics, and may be marketed at higher prices, thus increasing the profitability of the farming process. Using current algae growth techniques, approximately one acre of an algae pond 110 may generate the same amount of feed for cows as two acres of feed grown by conventional methods, further increasing the profitability of the farming process and also reducing its energy demands.

The illustration of FIG. 2 illustrates an overview of a methane convertor used in an ecosystem for methane conversion and emissions reduction. The methane produced 104 is driven into the methane convertor 106 by any appropriate means, which may include a fan 114 or a positive or negative pressure airflow system. The methane produced 104 is burned in the methane convertor 106 at a temperature, pressure, and rate that maintains the methane produced 104 below its combustion range. It is known that methane at concentrations over 5% becomes highly flammable and potentially explosive, so it is advantageous to regulate the concentration of methane produced 104 in the methane convertor 106. The methane produced 104 is passed across a heating means 116, such as a flame or a heating surface, and is converted into its products of combustion 108 within the methane convertor 106.

The methane convertor 106 may further comprise a plurality of sensors and controllers for accurately monitoring and displaying system status and methane concentrations. By way of example, a methane input sensor 118 may be installed in the methane convertor 106 before the heating means 116 so that it may measure methane concentrations before combustion. A methane output sensor 120 may be installed in the methane convertor 106 after the heating means 116 so that it may measure methane concentrations after combustion. The various sensors of the system may be controlled by a microprocessor 122 or any other appropriate electronic or mechanical means, and may further incorporate a user interface 124 for displaying system status and readings and receiving user inputs.

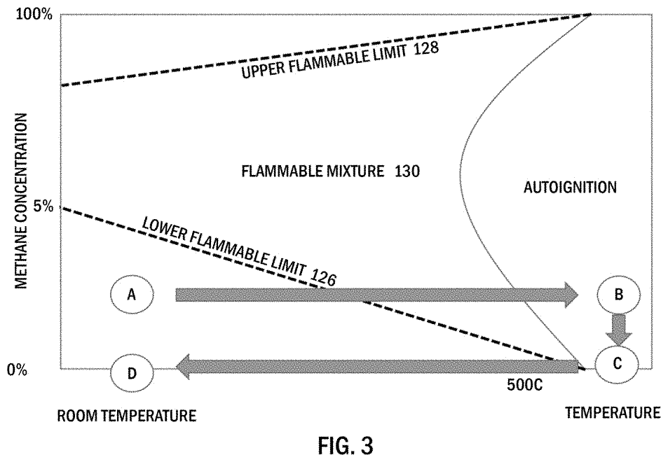

The illustration of FIG. 3 illustrates a process by which a methane convertor used in an ecosystem for methane conversion and emissions reduction operates. It is commonly known that if air with low concentrations of a combustible gas gets close to a flame, the gas in the air combusts without exploding. Fuel burns only when the fuel/air ratio 130 is within certain limits known as the flammable limits. In cases where fuels can form flammable mixtures with air, there is a minimum concentration of vapor in air below which propagation of flame does not occur. This is called the lower flammable limit 126. There is also a maximum concentration above which flame will not propagate called the upper flammable limit 128. These limits are generally expressed in terms of percentage by volume of vapor or gas in air.

The flammable limits change with temperature. By circulating the air close to the flame the methane-rich air is heated at the boundary of the flame, and, therefore, the flammable limits are reached even at 5% concentration or lower. Since the rest of the enclosed facility 102 is likely at room temperature the combustion of the methane will not propagate and, therefore, the system will be safe. The combustion of methane is very clean and is a primary reason for its use as a natural gas. Methane is used for cooking and heating even indoors because its reaction equation follows CH₄+2CO₂→CO₂+2H₂O.

Methane is less dense than ambient air and, thus, will rise to the top of the enclosed facility 102. Under certain conditions it may be possible to harvest the methane produced 104 from the top of the facility to light the heating means 116 in the methane converter 106. By utilizing this method, the cost of producing beef with be further reduced.

The table below compares the cost of farming by conventional methods against the cost of farming by the present invention. The cost of the traditional barn and the enclosed barns are very similar, and many large-scale farms already use enclosed barns. In current farms, it takes about 2 acres of land per cow to produce enough cow feed for the whole year in the United State. In the present invention the algae is fed with CO₂ produced by the system, which effectively results in twice the amount of feed per acre and lowering the cost of raising cattle. In 2017 US Dollars, the savings for the farmers will be about $100 per cow per year. The land used to grow feed rents, on average, at $70 per acre. In the present system it costs, on average, $31 per year to run the heating means 116, which may be further augmented by carbon credits earned from algae production and the recycling of methane produced 104 to drive the heating means 116.

TABLE 1

| Current Farming Method | Disclosed Method |
|---|---|
| Wood Barn | Inflatable barn |
| Fans | Fans |
| Rent 2 acres of land = $140 | Rent 1 acre shallow pond = $70 |
| | Running methane converter = $31 |
| | Eliminating methane CO2 marker |
| | 264 lb methane => 6,864 lb CO₂ = −$90 |
| Total Cost = $140 | Total Cost = $11 |

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims. Note with respect to the materials of construction, it is not desired nor intended to thereby unnecessarily limit the present invention by reason of such disclosure.

I claim:

1. An ecosystem for methane conversion and emissions reduction, comprising:
a plurality of ruminants;
an enclosed facility;
a methane convertor; and
an algae pond;
wherein said plurality of ruminants are contained within said enclosed facility;
wherein said plurality of ruminants discharge methane;
wherein said methane is contained within said enclosed facility;
wherein said methane is directed through said methane convertor;
wherein said methane convertor converts said methane into a plurality of products of combustion; and
wherein said plurality of products of combustion are transferred to said algae pond.

2. The invention of claim 1,
wherein said methane convertor further comprises a housing having a first portion, a second portion, and a means for combustion;
wherein said means for combustion is located between said first portion and said second portion of said methane convertor;
wherein said methane is directed through said first portion of said methane convertor; and
wherein said plurality of products of combustion are directed through said second portion of said methane convertor.

3. The invention of claim 2,
wherein said methane convertor further comprises a means for airflow;
wherein said means for airflow directs air from said enclosed facility into said first portion of said methane convertor;
wherein said means for airflow directs methane from said first portion of said methane convertor across said means for combustion;
wherein said means for combustion converts said methane into said plurality of products of combustion; and
wherein said means for airflow directs said products of combustion into said second portion of said methane convertor.

4. The invention of claim 3, further comprising:
an outlet path;
wherein said outlet path connects said second portion of said methane convertor to said algae pond; and
wherein said plurality of products of combustion are directed through said outlet path from said second portion of said methane convertor to said algae pond.

5. The invention of claim 4, further comprising:
a control system.

6. The invention of claim 5,
wherein said control system further comprises a plurality of sensors and a plurality of controllers;
wherein said plurality of sensors detect methane concentrations at a plurality of points within the system; and
wherein said plurality of controllers alter methane concentrations at a plurality of points within the system.

7. The invention of claim 6,
wherein said control system further comprises a user interface;
wherein said user interface displays a plurality of data parameters; and
wherein said user interface receives a plurality of user commands.

8. The invention of claim 7,
wherein said plurality of products of combustion are dissolved in said algae pond; and
wherein said plurality of products of combustion dissolved in said algae pond feeds a plurality of algae.

9. The invention of claim 8,
wherein said plurality of algae is fed to said plurality of ruminants.

10. The invention of claim 9,
wherein said means for combustion is a flame;

11. The invention of claim 10,
wherein said flame is fueled by said methane contained within said enclosed facility.

12. The invention of claim 9,
wherein said means for combustion is a hot surface.

13. The invention of claim 9,
wherein a plurality of animal waste is used to feed said plurality of algae.

14. The invention of claim 9,
wherein said plurality of products of combustion are used to feed an organic product.
